(19) United States
(12) Patent Application Publication
(10) Pub. No.: US 2018/0044738 A1
(43) Pub. Date: Feb. 15, 2018

(54) METHOD OF DIAGNOSING BREAST CANCER, MONITORING TREATMENT OF BREAST CANCER, AND TREATING BREAST CANCER

(71) Applicant: University Of Cincinnati, Cincinnati, OH (US)

(72) Inventor: Shao-Chun Wang, Mason, OH (US)

(21) Appl. No.: 15/677,899

(22) Filed: Aug. 15, 2017

Related U.S. Application Data
(60) Provisional application No. 62/375,125, filed on Aug. 15, 2016.

(51) Int. Cl. C12Q 1/68; A61K 31/506; A61K 31/517

(52) U.S. Cl. CPC C12Q 1/6886; A61K 31/517; A61K 31/506; C12Q 2600/118; C12Q 2600/178; C12Q 2600/158

(57) ABSTRACT

Described are methods of detecting circulating HOTAIR in breast cancer patients. The detection of circulating HOTAIR may aid in the treatment of triple-negative breast cancer (“TNBC”) or anti-estrogen resistant breast cancer. Circulating HOTAIR may be quantified before and after treatment to determine effectiveness of the treatment. Aspects are directed to diagnosing and treating TNBC and/or anti-estrogen resistant breast cancers in a subject by detecting circulating HOTAIR levels from a sample in the patient. The treatments may include administering agents that decrease the activity of EGFR and c-ABL, such as lapatinib and imatinib, respectively. Additional embodiments of the treatments include administration of a chimeric aptamer that targets both EGFR activity and HOTAIR expression.

### RELATED APPLICATIONS

This application is a non-provisional application claiming priority to U.S. Provisional Patent Application No. 62/375,9125 filed on Aug. 15, 2016 which is incorporated by reference in its entirety herein.

### FIELD

The present invention relates to methods of detecting circulating HOTAIR expression in breast cancer patients, and more particularly, to methods of detecting and decreasing HOTAIR expression in patients.

### BACKGROUND

Triple-negative breast cancer (TNBC), accounting for 10%-20% of breast cancer, is characterized by the lack of estrogen receptor (ER), and progesterone receptor (PgR), and low expression of the receptor tyrosine kinase ErbB2 (also known as HER2/neu). TNBC are often highly proliferative, of higher grade, and tend to be more aggressive than other types of breast cancer. Since TNBC is devoid of conventional therapeutic targets, it is the only major breast cancer type for which no specific FDA-approved targeted therapy is available. Radio- and chemo-therapies are the treatment options for TNBC. Thus, efficient targeted therapeutic regimens are urgently needed for TNBC.

EGFR is a receptor tyrosine kinase (RTK) of the ErbB family. Multiple signaling pathways, such as PI3K/AKT, mitogen-activated protein kinase (MAPK), and Wnt/P-catenin are activated by EGFR to enhance proliferation, survival, invasion, and metastasis of cancer cells. Expression of EGFR is frequently associated with TNBC and has been viewed as a promising therapeutic target. Unfortunately, the therapeutic efficacy of EGFR-targeting agents has been disappointing in breast cancer, suggesting that other molecular drivers also contribute to the malignancy. The non-receptor tyrosine kinase c-ABL promotes cell proliferation, migration, and survival. Multiple studies have demonstrated the important role of the non-receptor tyrosine kinase c-ABL in breast cancer. c-ABL expression is a frequent event in breast cancer and is associated with advanced tumor stages and metastasis. Combined treatment with lapatinib, a dual inhibitor of EGFR and ErbB2/HER2, and imatinib, a c-ABL inhibitor, results in synergistic growth inhibition in a panel of EGFR/ErbB2-expressing breast cancer cells including the TNBC cell line MDA-MB-468.

Long non-coding RNAs (lncRNAs) are non-coding RNAs greater than 200nt in length. Emerging evidence demonstrates the fundamental functions of lncRNAs in regulating genes associated with human diseases, including cancer. HOTAIR (HOX antisense intergenic RNA) is a 2.3 kb non-coding transcript derived from the intergenic region of the HOXC homeotic gene cluster. It functions as a scaffold to assemble epigenetic moderators to regulate gene expression. HOTAIR was the first lncRNA shown to promote tumor progression and is associated with poor prognosis in breast cancer. Expression of HOTAIR enhanced the growth and metastasis of xenograft tumors of mammary fat pad. However, virtually nothing is known about how this important LncRNA is regulated in cancer cells, or whether targeted therapeutic drugs affect its expression.

### SUMMARY

An embodiment of the invention is directed to a method of detecting changes in circulating HOTAIR in a patient. The method includes obtaining a circulatory sample at a first time point and quantifying the level of HOTAIR in the sample with polymerase chain reaction (“PCR”). At a later timepoint, another circulatory sample is obtained and the level of HOTAIR is quantify with PCR. The levels of HOTAIR in the two samples is compared to detect changes in HOTAIR expression between the two timepoints. In embodiment, a treatment may be administered to the patient between the collection of the two samples. The treatment may include administering agents that reduce the activity levels of epidermal growth factor receptor and c-ABL, such as, for example, a combination of lapatimib and imatinib, at a dose sufficient to reduce HOTAIR expression.

Another aspect of the invention is directed to diagnosing and treating TNBC or anti-estrogen resistant breast cancer in a patient by obtaining a circulatory sample from a patient and detecting the presence of HOTAIR in the sample. Diagnosing the patient with TNBC or anti-estrogen resistant breast cancer if HOTAIR is detected in the sample. A therapeutic agent is then administered to the patient in an amount effective to treat the TNBC or anti-estrogen resistant breast cancer.

Another aspect of the invention is directed to detecting the presence of TNBC or anti-estrogen resistant breast cancer in a patient. The method includes collecting a circulatory system sample from the patient and quantifying the level of HOTAIR in the sample.

### BRIEF DESCRIPTION OF THE DRAWINGS

The accompanying drawings, which are incorporated in and constitute a part of this specification, illustrate embodiments of the invention and, together with the general description of the invention given above and the detailed description of the embodiments given below, serve to explain the principles of the present invention.

FIG. 1 is schematic of representation of the molecular targets at the cellular level for detecting and treating TNBC or anti-estrogen resistant breast cancer in accordance with the principles of the present invention.

FIG. 2A is a bar graph showing the effect of dual treatment on exosomal expression of HOTAIR in cell culture experiment.

FIG. 2B is a bar graph showing the effect of dual treatment on exosomal expression of HOTAIR in another cell culture experiment.

FIG. 3 is a bar graph of data showing human HOTAIR may be found in exosomes from serum of mice with human tumor xenografts.

FIG. 4 is a bar graph of data showing the effect of treatments on the expression of human HOTAIR in exosomes from serum of mice with human tumor xenografts

FIG. 5A is a representation of an siRNA aptamer useful for treating TNBC and anti-estrogen resistant breast cancer in accordance with embodiments of the invention.

FIG. 5B is a graph showing the effect of the aptamer of FIG. 5A on HOTAIR expression in cells.

### DETAILED DESCRIPTION

Described are minimally invasive methods of detecting of a long non-coding RNA (“lncRNA”) called HOTAIR in a circulatory system sample from a patient. The patient may be a patient having a breast cancer diagnosis or symptoms indicating a diagnosis of breast cancer. In embodiments of the invention, the patient may have symptoms suggesting that the cancer is a TNBC or an anti-estrogen resistant breast cancer. Embodiments of the invention is directed to minimally invasive methods of diagnosing a subject with a TNBC or a breast cancer that is resistant to anti-estrogen treatments such as fulvestrant. A further aspect of the invention is directed to minimally invasive methods of monitoring the response to treatment of a subject having a TNBC or a breast cancer that is resistant to anti-estrogen therapeutics, such as fulvestrant. A further aspect of the invention is directed to treatments for TNBC and/or anti-estrogen resistant breast cancer that includes detecting HOTAIR in the subject and decreasing activity levels of EGFR and c-ABL. A further aspect of the invention is directed to treatments for TNBC and/or anti-estrogen resistant breast cancer that includes detecting HOTAIR in the subject and decreasing EGFR activity HOTAIR expression with a chimeric aptamer.

The methods include obtaining a sample from a patient, such as a sample from the patient's circulatory system that includes at least one of a serum sample, a blood sample, or a plasma sample from the patient. In an embodiment, the sample is a serum sample. In embodiment, the sample is obtained at a time point before anticancer treatments. In another embodiment, the sample is obtained at a time point after beginning anticancer treatment. In another embodiment, samples are obtained at a plurality of time points that may be before, during, and after the anticancer treatments.

In an embodiment, the presence of HOTAIR in the sample is detected by amplifying HOTAIR in the sample with polymerase chain reaction (“PCR”) and then detecting the amplified HOTAIR product. In an embodiment, the PCR is quantitative RT-PCR (“qRT-PCR”). Thus, in embodiments of the invention, detection of HOTAIR includes quantifying the level of HOTAIR expression in the sample. In embodiments, HOTAIR in the sample may be amplified with the following primers: forward GGTAGAAAAAGCAACCACGAAGC (SEQ ID NO: 1), and reverse ACATAAACCTCTGTCTGTGAGTGCC (SEQ ID NO: 2).

In embodiments of the invention, the level of HOTAIR detected in the sample is compared to a threshold level and a diagnosis of either having TNBC or breast cancer that is resistant to anti-estrogen therapeutics is based on the level of HOTAIR in the sample exceeding the threshold level. The threshold level can be based on the level of HOTAIR determined in populations of healthy subjects and populations of subjects having TNBC or breast cancer that is resistant to anti-estrogen therapeutics. When monitoring the treatment of a subject having TNBC or breast cancer that is resistant to anti-estrogen therapeutics, the level of HOTAIR is monitored for decreases in expression, or for a level of HOTAIR expression that is less than a threshold level, wherein the lowering levels indicate a positive response to treatment.

Anticancer treatments may include standard anticancer treatments as well as treatments that lower the activity levels of epidermal growth factor receptor (EGFR) and c-ABL. As discussed in greater detail below, in embodiments of the invention, the treatment includes a combination of lapatinib, which decreases the activity of EGFR, and imatinib, which decreases c-ABL activity. These drugs may be administered at a dose effective to decrease the activities of the respective EGFR and c-ABL pathways to synergistically decrease HOTAIR expression and cancer cell proliferation. In another embodiment, the anticancer treatment is a chimeric aptamer capable of targeting both EGFR activity and HOTAIR expression. An exemplary chimeric aptamer is discussed in greater detail below.

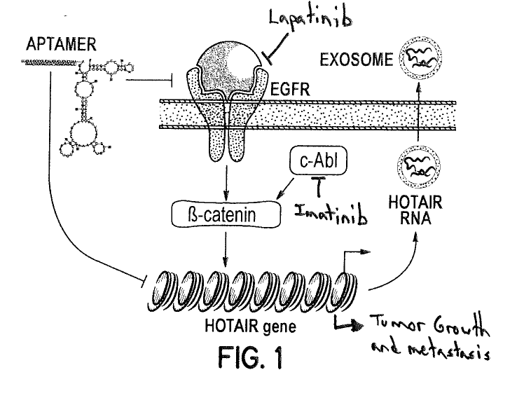

In a cohort of human TNBC cell lines, the receptor tyrosine kinase EGFR and the intracellular tyrosine kinase c-ABL independently promote nuclear expression of the oncoprotein J3-catenin which in turn form a complex with the transcription factor TCF4 to upregulate a long non-coding RNA called HOTAIR. Combined treatment of lapatinib and imatinib, FDA-approved inhibitor of EGFR and c-ABL, respectively, synergistically suppresses nuclear expression of J3-catenin and HOTAIR expression (FIG. 1). The EGFR-c-ABL-HOTAIR axis can be applied to breast cancer cells with developed resistance to the “pure anti-estrogen” fulvestrant which binds to ER and renders the receptor to degradation. It is noteworthy that breast cancer cells acquired resistance to fulvestrant are also depleted of ERa and PgR, and express elevated level of activated EGFR and c-ABL activities. Dual treatment with lapatinib and imatinib synergistically inhibits the growth of fulvestrant-resistant cells. These studies also demonstrate that HOTAIR lncRNA is secreted to the media from cultured cells.

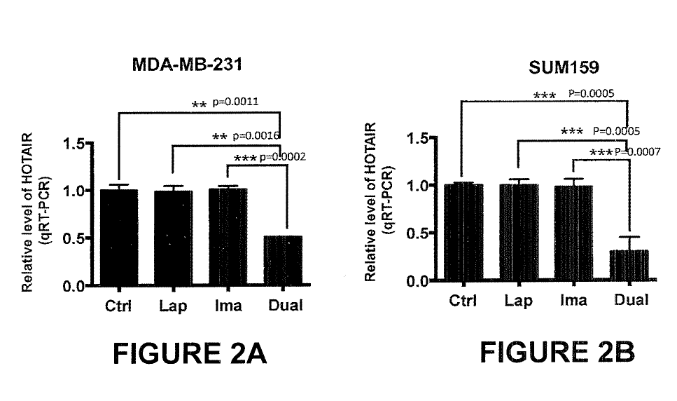

FIGS. 2A and 2B demonstrate that extracellular HOTAIR can be detected in the exosome compartment in the media of TNBC cells (MDA-MB-231 and SUM159). The level of exosome-associated HOTAIR is significantly reduced by dual treatment with lapatinib and imatinib, but not by either drug individually. To collect these data, TNBC cells were grown in culture and mock-treated or treated with lapatinib (5 μM for MDA-MB-231, 10 μM for SUM159) imatinib alone (10 μM for both cell lines), or the combination of agents (dual). Exosomes were isolated from the media and levels of HOTAIR were determined by quantitative RT-PCR.

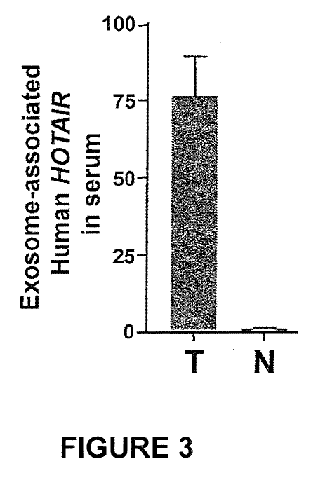

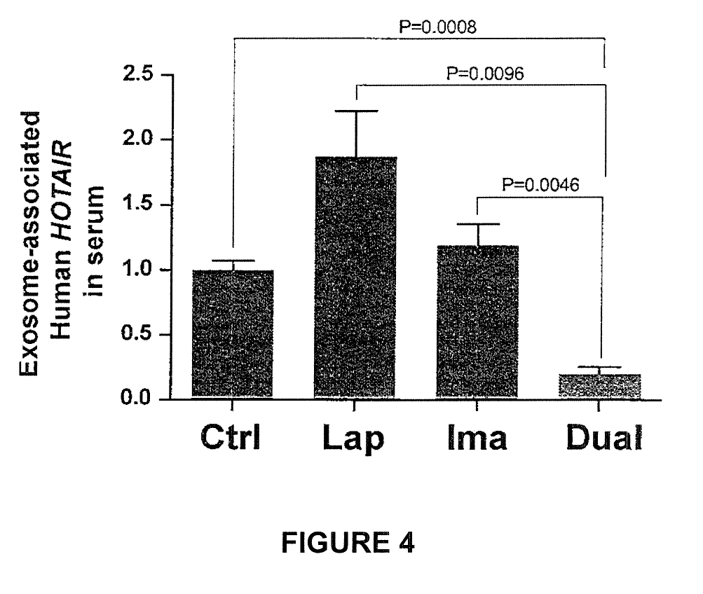

HOTAIR is secreted tumor tissues-derived exosomes and can be used as a circulatory biomarker in response to targeted treatment of cancer. Exosome are nanovesicles of 30-100 nm in diameter in size secreted from mammalian cells into bloodstream. Currently only few lncRNAs have been characterized as biomarkers in human fluids. As demonstrated in FIGS. 3 and 4, human HOTAIR RNA may be detected from exosomes isolated from the blood of mice bearing human tumor xenografts but not from the control mice without tumors. For these studies, human HOTAIR was detected in circulatory exosomes in tumor-bearing mice. HOTAIR RNA was measured by qRT-PCR using the ΔΔCₜ method and normalized to actin RND. T represents mice bearing MDA-MB-231-derived tumor xenografts; N represents mice without a xenograft. With respect to FIG. 4, these data were obtained from memory xenograft tumors derived from MDA-MB-231 that were treated with the indicated treatment (PBS control vehicle, lapatinib alone of 100 mg/kg, imatinib alone of 100 mg/kg, or the combination) (n+7 in each group). Serum exosomes were isolated two weeks after the treatment as describe for FIG. 3.

An embodiment of the present invention is directed to the use of exosome-associated long non-coding RNA HOTAIR as a circulatory biomarker in breast cancer. Another embodiment of the invention is directed to the use of exosomal HOTAIR as a circulatory prognostic marker associated with TNBC and/or advanced breast cancer, such as tumors developing resistance to anti-estrogens. In another embodiment, exosomal HOTAIR can be used as a marker of therapeutic responsiveness in dual treatment by imatinib and lapatinib. Thus, this biomarker is expected to have a wide application in the management of breast cancer.

Another aspect of the invention is directed to methods of diagnosing and treating TNBC and anti-estrogen resistant breast cancer in subjects with chimeric aptamers. An exemplary RNA aptamer is a 93-mer single-stranded nuclease-stabilized oligonucleotide. It has a dynamic three-dimensional conformation which specifically recognizes a target in a mode similar to antibodies but with high tissue penetration, low immunogenicity, and long in vivo stability. Aptamers targeting EGFR have been developed. The EGFR-specific aptamer E07 is a well characterized and potent anti-EGFR aptamer. This aptamer binds to EGFR with high affinity (K_{d}=2.4 nM), specifically targets EGFR-expressing breast cancer cells, blocks EGFR activity, and is internalized with EGFR. Described herein is an anti-EFGR aptamer (“aptEGFR”) developed using in vitro transcription which carries a phycoerythrin (PE)-conjugated oligonucleotide hybridized to its 3′ tail. The apt-EFGR is preferentially internalized by MDA-MB-231 cells but not EGFR-negative MCF-7 cells.

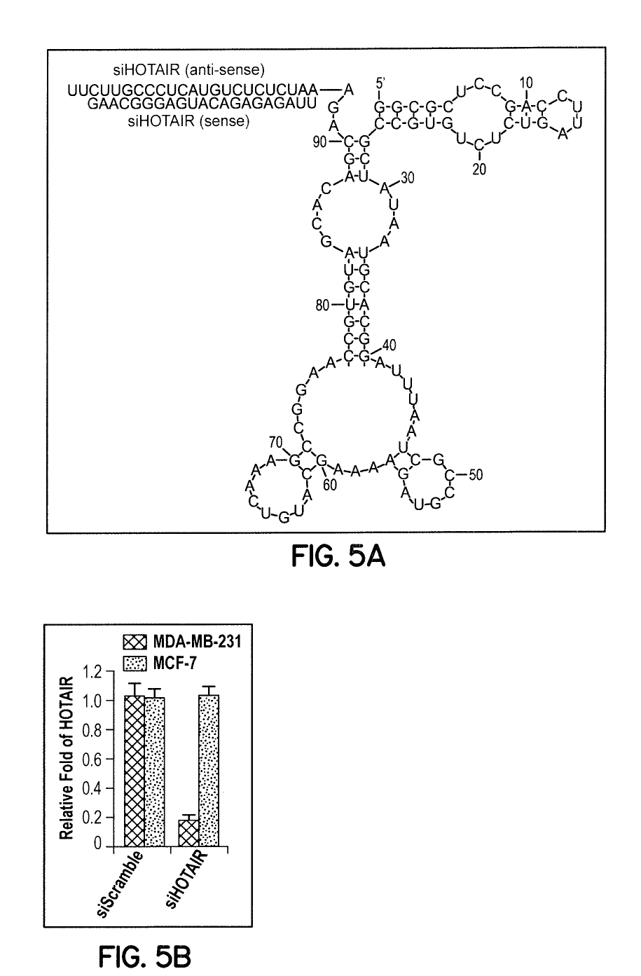

An exemplary aptEGFR-conjugated HOTAIR siRNA chimeric aptamer (aptEGFR-siHOTAIR), as depicted in FIG. 5A, was generated by fusing aptEGFR in cis with the 21-mer guide (antisense) strand of siHOTAIR. This aptamer, but not the control aptEGFR-siCtrl aptamer that is fused with scrambled siRNA, depleted endogenous HOTAIR in MDA-MB-231 cells but not that of MCF-7 cells (FIG. 5B), demonstrating the specificity and efficacy of the aptamer.

Additional chimeric aptamers with different siHOTAIRs may be generated from synthetic oligonucleotide templates and amplified by PCR with T7 primers followed by in vitro transcription with 2′-fluoropyrimidine modifications. The passenger strand (sense) of siHOTAIR may then be annealed to the aptamer body. These features promote the circulating half-life of the aptamer, and facilitate a conformation favoring Dicer recognition and loading to the RNA-induced silencing complex (RISC). Three more chimeric aptamers may be used as controls when testing the chimeric aptamers: aptEGFR-siCtrl—carrying a non-targeting siRNA of scrambled sequence (targets EGFR but not HOTAIR); aptCtrl-siHOTAIR—carrying a scrambled sequence in the random region of the aptamer (aptCtrl) and siHOTAIR (targets HOTAIR but not EGFR); aptCtrl-siCtrl—aptCtrl carrying a control siRNA. The most potent aptamer in vitro may be tested in the animal model.

A dose-response course of growth suppression activity of each aptamer may be established in a panel of EGFR-positive TNBC (MDA-MB-231, MDA-MB-468, BT-20, SUM159, HCC1806) and EGFR-negative (MCF-7, MDA-MB-453) cells (EGFR-negative TNBC cells lines are rare). All results will be normalized to basal growth inhibition by aptCtrl-siCtrl. It is expected that aptEGFR-siHOTAIR should repress HOTAIR expression and inhibit EGFR signaling by measuring EGFR kinase activity and phosphorylation of EGFR and MAPK. aptEGFR-siCtrl should inhibit EGFR but not HOTAIR, and have less growth inhibition activity than aptEGFR-siHOTAIR. The effects of growth inhibition will be assessed by 1) bromodeoxyuridine (BrdU) incorporation (proliferation), 2) annexin V staining and flow cytometry analysis (cell cycle and death), 3) soft agar assay (anchorage-independent cell growth), and 4) Boyd invasion chamber and wound healing assays (invasiveness).

Xenograft tumors derived from luciferase-expressing MDA-MB-231 and SUM159 cells will be implanted orthotopically in female nude mice. When tumors grow to −200 mm³, mice will be randomly assigned to the following six groups: a) aptEGFR-siHOTAIR, b) aptEGFR-siCtrl, c) aptCtrl-siHOTAIR, d) aptCtrl-siCtrl; e) lapatinib plus imatinib control; and 1) vehicle (corn oil) control. Initial dose of the aptamers will follow the previous animal studies (weekly intra-tumoral injection; 2000 pmol each time delivered in 10 sub-injections). The optimal dose will be determined empirically based on the effect on tumor volumes. The lapatinib plus imatinib group will be used to compare the tumor suppression efficacy of drug treatment with the aptamers. Based on preliminary results, oral gavage of 100 mg/kg of both drugs, but not either drug alone, synergistically suppresses tumor growth in a month (data not shown). Tumor volume will be measured weekly with a caliper, and also by an IVIS imaging system (Xenogen) which can reveal potential metastatic sites. Apoptosis will be assessed by TUNEL assay of the tumor tissues; proliferation will be assayed by in vivo BrdU incorporation analysis. Primary tumor with adjacent normal tissues will be examined by hematoxylin/eosin (H&E) staining. Tumor invasion will be assessed based on the extent of infiltration into surrounding tissues, the margins from surrounding tissues, and desomoplastic stromal reaction by smooth muscle actin staining. Human origin of the invasive cells will be confirmed by staining for human cytokeratin.

While the present invention has been illustrated by the description of embodiments thereof, and while the embodiments have been described in considerable detail, it is not intended to restrict or in any way limit the scope of the appended claims to such detail. Additional advantages and modifications will readily appear to those skilled in the art. The invention in its broader aspects is, therefore, not limited to the specific details, representative apparatus and method, and illustrative examples shown and described. Accordingly, departures may be made from such details without departing from the spirit or scope of the general inventive concept.

Having described the invention, what is claimed is:

1. A method of detecting changes in circulating HOTAIR in a patient, said method comprising:
obtaining a first circulatory sample from a patient at a first time point;
quantifying a level of circulating HOTAIR in the first circulatory sample by amplifying HOTAIR in the first circulatory sample with a polymerase chain reaction;
obtaining a second circulatory sample from the patient at a second time point;
quantifying a level of circulating HOTAIR in the second circulatory sample by amplifying HOTAIR in the second circulatory sample with polymerase chain reaction; and
comparing the level of circulating HOTAIR quantified in the first circulatory sample with the level of circulating HOTAIR in the second circulatory sample to detect changes in the circulating HOTAIR between the first and second circulatory samples.

2. The method of claim 1 wherein the first circulatory sample is selected from the group consisting of a first blood sample, a first serum sample, a first plasma sample, and combinations thereof and the second circulatory sample is a second blood sample, a second serum sample, a second plasma sample, and combinations thereof.

3. The method of claim 1 wherein the first circulatory sample is a first serum sample and the second circulatory sample is a second serum sample.

4. The method of claim 1 further comprising administering a treatment to said patient after obtaining said first circulatory sample and before obtaining said second circulatory sample.

5. The method of claim 1 wherein the treatment includes administering to the patient an amount of a therapeutic agent or combination of therapeutic agents effective to decrease the activity level of each of epidermal growth factor receptor (“EGFR”) and c-ABL.

6. The method of claim 1 wherein the therapeutic treatment includes administering to the patient an amount of each of lapatinib and imatinib, in combination, effective to decrease the activity level of each of EGFR and c-ABL.

7. The method of claim 1 wherein the therapeutic treatment includes administering to the patient an amount of each of lapatinib and imatinib, in combination, effective to decrease the expression of HOTAIR in the second circulatory sample relative to the first circulatory sample.

8. The method of claim 1 wherein the therapeutic treatment includes administering to the patient an amount of a chimeric aptamer to decrease the expression of HOTAIR in the second circulatory sample, wherein said chimeric aptamer targets EGFR activity and HOTAIR expression.

9. The method of claim 1 wherein the HOTAIR is amplified in a quantitative RT-PCR reaction.

10. The method of claim 1 wherein the HOTAIR is amplified with primers have SEQ ID NO. 1 and SEQ ID NO: 2.

11. A method of diagnosing and treating triple-negative breast cancer (“TNBC”) in a patient, the method comprising:
obtaining a sample from a human patient; and
detecting whether HOTAIR is present in the sample by amplifying HOTAIR in the serum sample with a polymerase chain reaction and detecting the amplified HOTAIR;
diagnosing the patient with TNBC when HOTAIR is present in the sample; and
administering to the patient an amount of a therapeutic agent or combination of therapeutic agents effective to treat the TNBC.

12. The method of claim 11 wherein the therapeutic treatment includes administer to the patient an amount of each lapatinib and imatinib, in combination, effective to decrease the expression of HOTAIR in the serum of the patient.

13. The method of claim 11 wherein the therapeutic treatment includes administer to the patient an amount of each lapatinib and imatinib, in combination, effective to decrease the expression of HOTAIR in the serum of the patient.

14. The method of claim 11 wherein the therapeutic agent or combination therapeutic agents includes an amount of each of lapatinib and imatinib, in combination, effective to decrease the activity level of each of EGFR and c-ABL.

15. The method of claim 11 wherein the therapeutic agent or combination therapeutic agents includes an amount of each of lapatinib and imatinib, in combination, effective to decrease the expression of HOTAIR in the second sample relative to the first sample.

16. The method of claim 11 wherein the therapeutic agent or combination of therapeutic agents is a chimeric aptamer capable of targeting EGFR activity and HOTAIR expression.

17. The method of claim 11 wherein the HOTAIR is amplified in a quantitative RT-PCR reaction.

18. The method of claim 11 wherein the HOTAIR is amplified with primers have SEQ ID NO. 1 and SEQ ID NO: 2.

19. A method of detecting the presence of triple-negative breast cancer (“TNBC”) or the presence of anti-estrogen resistant breast cancer in a patient, the method comprising:
obtaining a circulatory sample from a human patient;
quantifying a level of HOTAIR in the circulatory sample by amplifying exosomal HOTAIR using qRT-PCR to detect the presence of at least one of TNBC or anti-estrogen resistant breast cancer if the level of HOTAIR in the circulatory system is above a threshold.

20. The method of claim 19 wherein the HOTAIR is amplified with primers have SEQ ID NO. 1 and SEQ ID NO: 2.


SEQUENCE LISTING (3 sequences)

SEQ ID NO: 1 - DNA, 23 bp, Artificial Sequence
  Synthesized
ggtagaaaaagcaaccacgaagc

SEQ ID NO: 2 - DNA, 25 bp, Artificial Sequence
  Synthesized
acataaacctctgtctgtgagtgcc

SEQ ID NO: 3 - DNA, 296 bp, Artificial Sequence
  Synthesized
tggctttagctcctacattaaggctcggacctagtcctcctgatcaaaggagctcgcggcatttttatgagatcaacttcagcaactctttattagcgatcagatgcctgggtcgaaaatgcaaaagggaacaaacagctaatcaatccaaaaggaaaaagaaaagagaggggtgggaaggcatggggtgaaaaacttcaggtgacacaaagcaaaaagattccagtcaccaaaaacaaaaacaaaaaaaaatcttttgcccccagcaagaatcatttgtttgggatctgttccag